(19) United States
(12) Patent Application Publication
(10) Pub. No.: US 2008/0108716 A1
(43) Pub. Date: May 8, 2008

(54) Simple low-pressure fischer-tropsch process

(76) Inventor: Conrad Ayasse, Calgary (CA)

Correspondence Address: GOWLING LAFLEUR HENDERSON LLP, SUITE 1400, 700 2ND ST. SW, CALGARY, AB, T2P 4V5, omitted

(21) Appl. No.: 11/594,209

(22) Filed: Nov. 8, 2006

(51) Int. Cl. C07C 27/06

(52) U.S. Cl. 518/715

(57) ABSTRACT

A process for combing carbon monoxide and hydrogen to form liquid fuels such as diesels, and/or waxes, in a single pass fixed bed reactor, at an operation pressure less than 200 psig. The reactor uses a catalyst with a metallic cobalt loading greater than 5% by weight and a rhenium loading of less than 2% by weight, on an alumina support.

### FIELD OF THE INVENTION

This invention related to a process for converting carbon monoxide and hydrogen to liquid motor fuels and/or waxes, and in particular a low pressure process using a particular catalyst on a defined substrate.

### BACKGROUND

i) Overview

The Fischer-Tropsch (FT) process for converting carbon monoxide and hydrogen to liquid motor fuels and/or wax has been known since the 1920's.

During the Second World War synthetic diesel was manufactured in Germany using coal gasification to supply a 1:1 ratio of hydrogen and carbon monoxide for conversion to fuel hydrocarbons. Because of trade sanctions and the paucity of natural gas, South Africa further developed the coal via gasification route to synthesis gas and employed a fixed-bed iron Fischer-Tropsch catalyst. Iron catalysts are very active for the water-gas shift reaction which moves the gas composition from a deficiency of hydrogen and closer to the optimum H₂/CO ratio of around 2.0. When large natural gas supplies were developed, steam and autothermal reformers were employed to produce the synthesis gas feedstock to slurry-bed FT reactors using cobalt or iron catalysts.

In Gas-To-Liquids (GTL) plants, compromises must be made between liquid product yield and plant operating and capital costs. For example, if there is a market for electricity, a steam reformer design may be chosen because this technology produces a large amount of waste heat: flue gas heat can be converted to electricity using an ‘economiser’ and steam turbine. If conservation of natural gas feedstock and low capital cost are paramount, autothermal or partial oxidation reformers using air are favored.

Another factor in selecting the best reformer type is the nature of the reformer hydrocarbon feed gas. If the gas is rich in CO₂, this can be advantageous because the desired H₂/CO ratio can then be achieved directly in the reformer gas without the need to remove excess hydrogen, and some of the CO₂ is converted to CO, increasing the potential volume of liquid hydrocarbon product that can be produced. Additionally, the volume of steam that is required is reduced, which reduces the process energy requirements,

The market for Fischer-Tropsch (FT) processes is concentrated on large “World-Scale” plants with natural gas feed rates of greater than 200 million scfd because of the considerable economies if scale. These plants operate at high-pressure, about 450 psia, and use extensive recycling of tail gas in the FT reactor. For, example, the Norsk Hydro plant design has a recycle ratio of about 3.0. The emphasis is on achieving the maximum wax yield. In terms of product slate, these large plants strive for the maximum yield of FT waxes in order to minimize the formation of C₁-C₅ products. The waxes are then hydrocracked to primarily diesel and naphtha fractions. Unfortunately, light hydrocarbons are also formed in this process. The reformers use some form of autothermal reforming with oxygen produced cryogenically from air, an expensive process in terms of operating cost and capital cost. The economies of scale justify the use of high operating pressure, the use of oxygen natural gas reforming, extensive tail gas recycling to the FT reactor for increasing synthesis gas conversion and controlling heat removal and product wax hydrocracking. To date, an economical FT plant design has not been developed for small plants with capacities of less than 100 million scfd.

The present invention strives for optimized economics in a completely different market: small FT plants using less than 100 million scfd. The emphasis is on simplicity and minimized capital cost, somewhat at the expense of efficiency.

| ii) Existing FT Technologies | Technology of this invention |
|---|---|
| Large plants, >25 MMscfd | Small plants, <100 MMscfd |
| High pressure, >200 psia | Low pressure, <200 psia |
| Oxygen to reformer | Air to reformer |
| Extensive recycling to FT | No recycling (“once-through” |
| reactor or reformer | process) |
| Low single-pass FT CO | High single-pass conversion (>65%) |
| conversion (<50%) | |
| Deliberate and extensive | Less than 10% wax formation |
| wax formation | |
| Hydrocracking waxes | No hydrocracking operations |
| Multiple-pass FT reactors | Single-pass-FT reactor |
| Low FT diesel yield (<50%) | High diesel yield (55–90% of |
| | hydrocarbon liquid) |

iii) Prior Art

The catalytic hydrogenation of carbon monoxide to produce a variety of products ranging from methane to heavy hydrocarbons (up to C₈₀ and higher) as well as oxygenated hydrocarbons is usually referred to as Fischer-Tropsch synthesis. The high molecular weight hydrocarbon product primarily comprises normal paraffins which can not be used directly as motor fuels because their cold properties are not compatible. After further hydroprocessing, Fischer-Tropsch hydrocarbon products can be transformed into products with a higher added value such as diesel, jet fuel or kerosene. Consequently, it is desirable to maximize the production of high value liquid hydrocarbons directly.

Catalytically active group VIII, in particular, iron, cobalt and nickel are used as Fischer-Tropsch catalysts; cobalt/ruthenium is one of the most common catalyzing systems. Further, the catalyst usually contains a support or carrier metal as well as a promoter, e.g., rhenium.

Metal oxides, e.g., silica, alumina, titania, zirconia or mixture thereof, have been utilized as catalyst supports in Fischer-Tropsch hydrocarbons synthesis. U.S. Pat. No. 4,542,122 disclosed a cobalt or cobalt thoria on titania as a hydrocarbon synthesis catalyst. U.S. Pat. No. 4,088,671 disclosed a cobalt-ruthenium catalyst where alumina was used as a support. European Pat. No. 142,887 described a silica supported cobalt catalyst together with zirconium, titanium, ruthenium and/or chromium.

U.S. Pat. No. 4,801,573 described a promoted cobalt and rhenium catalyst supported on alumina. The amount of cobalt is most preferably about 10-40 wt % of the catalyst. However, rhenium is preferably about 2-20 wt of cobalt content. U.S. Pat. No. 5,248,701 disclosed a copper promoted cobalt-manganese spinel that was said to be useful as a Fischer-Tropsch catalyst with selectivity for olefins and higher paraffins.

U.S. Pat. No. 4,738,948, issued in Apr. 19, 1988, describes a catalyst comprising cobalt ruthenium at an atomic ratio of 10-400, on a refractory carrier, such as titania or silica. The catalyst is used for conversion of synthesis gas with an H₂:CO ratio of 0.5-10, preferably 0.5-4, to C₅-C₄₀ hydrocarbons at a pressure of 80-600 psig and at a temperature of 160-300° C., at a gas hourly space velocity of 100-5000 v/hr/v.

After a period of time in operation, a catalyst becomes deactivated, losing its effectiveness for synthesis gas conversion. Among the main deactivation mechanisms for cobalt based catalysts are sulfur poisoning [e.g. R. L. Espinoza, et al, Applied Catalysis A:General 186 (1999)13], metal oxidation [e.g. D. Schanke et al, Catal. Lett. 34 (1995) 269] and surface condensation of heavy hydrocarbons [e.g. E. Iglesia et al, J. Catal. 143 (1993)345].

U.S. Pat. No. 5,728,918, issued on Mar. 17, 1998, described a catalyst comprising cobalt on a support, used for conversion of synthesis gas with an H₂:CO ratio of 1-3, preferably 1.8-2.2, to C₅⁺ hydrocarbons at a pressure of 1-100 bar and at a temperature of 150-300° C., at a typical gas hourly space velocity of 1000-6000 v/hr/v. Generation Activation of this catalyst was achieved by using a gas containing carbon monoxide and less than 30% hydrogen at a temperature more than 10° C. above Fischer-Tropsch conditions in the range 100-500° C. at a pressure of 0.5-10 bar, for air, at least 10 min preferably 1-12 hours.

Fischer-Tropsch synthesis performed at low pressure, 17-21 atmospheres, and relatively high temperature, usually produces short chain hydrocarbons of 0.6-0.7 chain growth probability factor. U.S. patent application Ser. No. 20050209348 issued on Sep. 22, 2005, described a Fischer-Tropsch process performed at an elevated temperature between 230-280° C., for example 240° C. and at elevated pressure typically between 1.7 MPa and 2.1 MPa, for example 1.8 MPa, using a compact reactor. The preferred catalyst comprised a coating of gamma alumina support with 10-40% cobalt (by weight compared to the alumina) and with a promoter such as ruthenium, platinum or gadolinium which is less than 10% of the cobalt weight. The gas hourly space velocity was very high, for example 20000 hr⁻¹ and the produced hydrocarbon liquid consisted of saturated linear alkanes of chain lengths range between about 6-17. Consequently, it is rich in aircraft fuel. However, the selectivity to the production of C₅⁺ hydrocarbon was less than 65% and the conversion of carbon monoxide was no greater than 75%.

Hence, there is still a great need to identify other Fischer-Tropsch processes which can be used to directly produce different types of fuel such as, diesel fuel.

### SUMMARY OF THE INVENTION

The invention consists of a low-pressure Fischer-Tropsch process and a catalyst that produces a high diesel-fraction yield. Process pressure is below 200 psig. The catalyst is cobalt deposited at greater than 5 weight percent on gamma alumina, optionally along with rhenium or ruthenium at 0.01-2 wt. %. It has been discovered that this catalyst is very effective at low pressures in converting synthesis gas into diesel in high yield. The present invention is particularly well suited to conversion of low pressure gases containing low molecular weight hydrocarbons into FT liquids. Examples of applications are landfill gas, oil field solution gas and low pressure gas from de-pressured gas fields. In all these cases, multiple-stage gas and air compression would be required in traditional FT plants. The high efficiency of the present FT catalyst enables high CO conversion and produces a product stream containing up to 90+ wt. % diesel in a single pass. The use of air in the natural gas reformer provides a synthesis gas containing approximately 50% nitrogen, which facilitates heat removal in the FT reactor as sensible heat and increases gas velocity and heat transfer efficiency, so that tail gas recycling is not needed. Naphtha can be partially separated from the hydrocarbon product by flash distillation at low cost to generate a more pure diesel product. This also serves to provide some product cooling. The liquid hydrocarbon product is excellent for blending with petroleum diesel to increase cetane number and reduce sulfur content.

### BRIEF DESCRIPTION OF THE DRAWINGS

FIG. 1 is a process flow diagram for a particular embodiment of the invention;

FIG. 2 is a flow diagram for flash separation of naphtha and diesel hydrocarbon fractions as a subsequent step to the Fischer-Tropsch process of the present invention;

FIG. 3 is a graph showing the effect of pressure on catalyst performance, for a catalyst of Example 4 at 202.5° C.;

FIG. 4 is a graph showing the effect of carbon number on the % weight production of liquid fuels and/or waxes, at 190° C., 70 psia, using a CSS-350 alumina support (ie Example 7), for a catalyst of example 4 at 202.5° C.;

FIG. 5 is a graph showing the effect of carbon number distribution on the % weight production of liquid fuels and/or waxes, at 220° C. and 70 psia, using an LD-5 alumina support (ie. Example 8);

FIG. 6 is a graph showing the effect of carbon number distribution on the % weight production of liquid fuels and/or waxes, at 190° C. at 70 spia using an F-220 alumina support (ie Example 9);

FIG. 7 is a graph showing the effect of carbon number distribution on the % weight production of liquid fuels and/or waxes, for the catalyst of for Example 3 at 190° C.;

FIG. 8 is a graph showing the effect of carbon number distribution on the % weight production of liquid fuels and/or waxes, for the catalyst of Example 10 using ruthenium promoter;

FIG. 9 is a graph showing the effect of carbon number distribution on the % weight production of liquid fuels and/or waxes, for ruthenium promoter and LD-5 alumina support, using an Aerolyst 3038 silica catalyst support instead of alumina;

FIG. 10 is a graph showing a comparison of Example 9 carbon distribution with a traditional Anderson-Shultz-Flory distribution.

### DETAILED DESCRIPTION OF THE PREFERRED EMBODIMENT

In order to operate the FT process at high conversions with oxygen-blown reformer synthesis gas, the approach has been to recycle tail gas in a high proportion- at a ratio of 3.0 or greater bases on fresh gas feed. A secondary benefit is that the fresh gas is diluted in carbon monoxide, which reduces the required rate of heat removal from the FT reactor, reduces hot-spotting and improving the product slate. However, tailgas recycling is a very energy and capital expensive activity. The separation of oxygen from air is also an energy and capital expensive activity.

The approach taken in the present process is to use air in the reformer, which gives a synthesis gas containing approximately 50% nitrogen as inert diluent, eliminating the need for tail gas recycling to moderate FT reactor heat removal requirements. Others employing air-blown synthesis gas in FT processes have achieved the desired high CO conversions by using multiple FT reactors in series, which entails high capital costs and complex operation. The present process achieves high CO conversion in a simple single pass and a high diesel cut by using a special catalyst.

The catalyst of the present invention employs an alumina support with high cobalt concentration, along with a low level of rhenium to facilitate catalyst reduction. The high cobalt concentrations increase catalyst activity, enabling high single-pass synthesis gas conversion.

The Anderson-Shultz-Florey theory predicts the FT hydrocarbons to cover a very wide range of carbon numbers, from 1-60, whereas the most desireable product is diesel fuel (C₉-C₂₃, Chevron definition). In order to reduce the ‘losses’ of CO to making C₁-C₅ hydrocarbons, a common approach is to strive to make mostly wax in the FT reactor and then, in a separate operation, to hydrocrack the wax to mostly diesel and naphtha. Surprisingly, the process and catalyst of the present invention makes diesel in high yield (to 90 wt %) directly in the FT reactor, obviating the need for expensive and complex hydrocracking facilities.

Because of the elimination of oxygen purification, high-pressure compression, tail gas recycling and hydrocracking, the present process can be applied economically in much smaller plants than hitherto considered possible for FT technology.

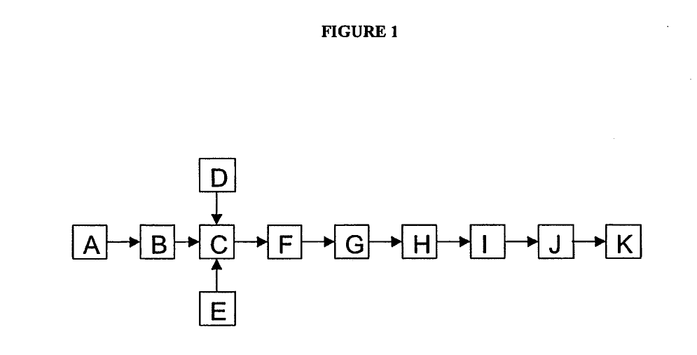

FIG. 1 shows the process flow diagram for the present invention, wherein the letters A-k signify the following:
A Raw hydrocarbon-containing gas
B Gas conditioning equipment
C Reformer
D Water
E Air or oxygen
F Cooler
G Water removal
H hydrogen removal (optional)
I Fischer Tropsch reactor
J Back-pressure regulator
K Product cooling and recovery (2-options)

Letter A represents the raw hydrocarbon-containing process feed gas. This could be from a wide variety of sources: for example, from a natural gas field, a land-fill facility (biogenic gas), a petroleum oil processing facility (solution gas), among others. The pressure of the gas for the present process can vary widely, from atmospheric pressure to 200 psia or higher. Single-stage or two-stage compression may be required, depending on the source pressure and the desired process operating pressure. For example, for landfill gas, the pressure is typically close to atmospheric pressure and blowers are used to transmit the gas into combustion equipment. Solution gas, which is normally flared, must also be compressed to the process operating pressure. There are also many old exploited and late-life natural gas fields with pressure too low for acceptance into pipelines that could make possible feedstock for the present process. Other natural gas sources, which may or may not be stranded (no access to a pipeline) may already be at or above the desired process operation pressure and these are also candidates. Another candidate is natural gas that is too high in inerts such as nitrogen to meet pipeline specifications.

Letter B represents hydrocarbon gas conditioning equipment. The gas may require clean-up to remove components that would damage reformer or FT catalyst. Examples of these are mercury, hydrogen sulfide, silicones and organic chlorides. Organic chlorides, such as found in land-fill gas, produce hydrochloric acid in the reformer, which can cause severe corrosion. Silicones form a continuous silicon dioxide coating on the catalyst, blocking pores. Hydrogen sulphide is a powerful FT catalyst poison and is usually removed to 1.0 ppm or lower. Some gas, from sweet-gas fields, may not require any conditioning (clean-up).

The hydrocarbon concentration in the raw gas affects the economics of the process because less hydrocarbon product is formed from the same volume of feed gas. Nevertheless, the process can operate with 50% or lower methane concentration, for example, using land-fill gas. There may even be reasons to operate the process even at a financial loss: for example to meet greenhouse gas government or corporate emission standards. The process can operate with feed gases containing only methane hydrocarbon or containing natural gas liquids by the application of known reformer technologies. The presence of carbon dioxide in the feed gas is beneficial.

Letter C represents the reformer, which may be of several types depending on the composition of the feed gas. A significant benefit of low pressure reformer operation is the lower rate of the Brouard reaction and diminution of metal dusting.

Partial oxidation reformers normally operate at very high pressure ie. 450 psia or greater, and so are not optimum for a low-pressure FT process. It is energetically inefficient, and can easily make soot, however, it does not require water, and makes a syngas with a H₂/CO ratio near 2.0, optimum for FT catalysts. Partial oxidation reformers may be employed in the present process.

Steam reformers are capital expensive and require flue gas heat recovery to maximize efficiency in large plants. Because the synthesis gas contains relatively low levels of inerts such as nitrogen, temperature control in the FT reactor can be difficult without tail gas recycling to the FT reactor. However, the low level of inerts enables recycling of some tail gas to the reformer tube-side, supplementing natural gas feed, or to the shell side to provide heat. Keeping in mind that FT tail gas must be combusted before venting in any event, this energy can be used for electrical generation or, better yet, to provide the reformer heat which would be otherwise be provided from burning natural gas. For small FT plants, steam reformers are a viable choice. Steam reformers may be employed in the present process.

Autothermal reforming is an efficient process of relatively low capital cost that uses moderate temperatures and modest steam concentrations to produce a soot-free synthesis gas with H₂/CO around 2.5 using low-CO₂ natural gas feed, which is closer to the desired ratio than for steam reforming. However some hydrogen removal is still required for most natural gas feeds. If the feed gas contains greater than about 33% CO₂, as is the case with land-fill gas feed, then an H₂/CO ratio of 2.0 can be achieved without any recycle streams, and the water use can also be diminished. This is the most desired type of reformer for the present low-pressure FT processes.

Letter D represents the optional water that is injected as steam into the reformer. All reformer technologies except partial oxidation require the injection of steam.

Letter E represents an oxidizing gas, which could be air, oxygen or oxygen-enriched air.

Letter F represents a cooler for reducing the reformer outlet temperature from greater than 800° C. to close to ambient. The cooling may be done in several stages, but preferably in a single stage. The cooling may be achieved with shell-and-tube or plate-and-frame heat exchangers and the recovered energy may be utilized to pre-heat the reformer feed gases, as is well known in the industry. Another way of cooling the reformer tail gas is by direct injection of water into the stream or by passing the stream through water in a vessel.

Letter G represents a separator for separating the reformer synthesis gas from condensed water, so as to minimize the amount of water entering downstream equipment.

Letter H represents optional hydrogen removal equipment such as Prism™ hydrogen-selective membranes which are sold by Air Products, or Cynara membranes from Natco.

Certain reformer processes produce a synthesis gas too rich in hydrogen, some of which must be removed to achieve optimum FT reactor performance. An ideal H₂/CO ratio is 2.0-2.1, whereas the raw synthesis gas may have a ratio of 3.0 or higher. High hydrogen concentrations give rise to larger CO loss to producing methane instead of the desired motor fuels or motor fuel precursor such as naphtha.

Letter I represents typical FT reactors, which are of the fixed-bed or slurry bubble type and either may be used. However, the fixed-bed is preferred because if its simplicity of operation and ease of scale-up.

Letter J represents a back-pressure controller which sets the process pressure. It may be placed in other locations depending on the product recovery and possible partial separation process employed.

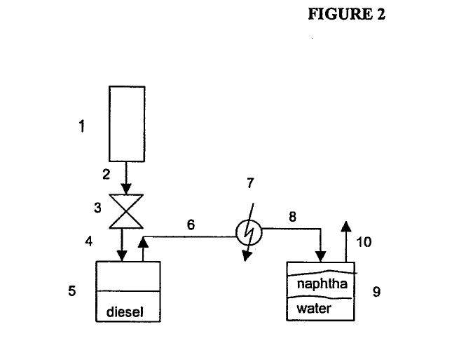

Letter K represents product cooling and recovery. Product cooling is typically accomplished by heat exchange with cold water and serves to pre-heat the water for use elsewhere in the FT plant. Separation is accomplished in a separator vessel designed for oil/water separation. However a second alternative is to flash-cool the FT reactor product before the aforementioned cooler-separator as shown in FIG. 2. This serves two purposes-firstly to reduce the product temperature and secondly to enable partial separation of the naphtha component in the produced hydrocarbon product, enriching the remaining liquid in the diesel component.

FIG. 2 shows a process diagram, for flash separation of naphtha and diesel hydrocarbons, in which:
1 is a fixed-bed Fischer Tropsch reactor.
2 is a mixture of gases, water, naphtha, diesel and light waxes at ca. 190-240° C. and pressure greater than atmospheric.
3 is a pressure let-down valve.
4 is stream 2 at reduced temperature due to gas expansion and at 14.7 psia.
5 is a flash drum vessel.
6 is a vapour phase consisting of stream 2 minus diesel and light waxes.
7 is a cooler.
8 is stream 6 with naphtha and water in the liquid phase.
9 is a vessel to retain naphtha and water.
10 is a waste tailgas stream consisting mainly of inert gases and light hydrocarbons.

The FT products 2 flow through a pressure let-down valve 3 and into a flash drum 5. The inert gases and lower-boiling hydrocarbons, water and naphtha go overhead as vapour out of the flash drum and through cooler 7. The diesel and light waxes collect in vessel 5. The water and naphtha condense in cooler 7 and are collected in vessel 9. The remaining gases exit overhead in stream 10 and are typically combusted, sometimes with energy recovery, or are used to generate electricity.

### EXAMPLES

TABLE 1
Physical characteristics of alumina supports.

| | | Alcoa | | |
|---|---|---|---|---|
| Alumina catalyst | Alcoa | CSS- | Alcoa | Sasol |
| support | LD-5 | 350 | F-220 | (Trilobes) |
| Surface Area, m²/g | 300 min | 350 | 360 | 248 |
| Pore Volume, cc/g | 0.63 | 0.57 | 0.5 | 0.82 |
| Bulk Density, g/cc | 0.465 | 0.72 | 0.769 | 0.42 |
| Al₂O₃, % wt | Diff. | 99.6 | 93.1 | Diff. |
| SiO₂, % wt | 0.40 | 0.02 | 0.02 | 0.015 |
| | max | | | |
| Fe₂O₃, % wt, max | 0.04 | — | 0.02 | 0.015 |
| Na₂O, % wt, max | 0.2 | 0.35 | 0.3 | 0.05 |
| LOI (250–1100° C.), % wt | 23–30 | 3.5 | 6.5 | — |

### Example 1

Catalyst synthesis was conducted by ordinary means as practiced by those knowledgeable in the art. The catalyst support was alumina trilobe extrudate obtained from Sasol Germany GmbH (hereafter referred to as ‘trilobe’) The extrudate dimensions were 1.67 mm diameter and 4.1 mm length. The support was calcined in air at 500° C. for 24 hours. A solution mixture of cobalt nitrate and perrhenic acid was added to the support by the method of incipient wetness to achieve 5 wt % cobalt metal and 0.5 wt. % rhenium metal in the finished catalyst. The catalyst was dried slowly and then heated in a convection oven at the rate of 1.0° C. per minute to 350° C. and held at that temperature for 12 hours. A volume of 29 cc of oxidized catalyst was placed in a ½ inch OD tube that had an outer annular space through which temperature-control water was flowed under pressure in order to remove the heat of reaction. In effect, the FT reactor was a shell-and-tube heat exchanger with catalyst placed in the tube side. The inlet gas and water were both at the targeted reaction temperature. Catalyst reduction was accomplished by the following procedure:

Reduction-gas flow rate (cc/min)/H₂ in nitrogen (%)/temperature (° C.)/time (hours):

1. 386/70/200/4, pre-heat stage

2. 386/80/to 325/4, slow heating stage

3. 386/80/325/30, fixed-temperature stage

During Fischer-Tropsch catalysis, total gas flow to the FT reactor was at a GHSV of 1000 hr⁻¹. Gas composition was representative of an air-autothermal reformer gas: 50% nitrogen, 33.3% H₂ and 16.7% CO. A seasoning of the catalyst was used to reduce methane production. This was accomplished by holding the reactor temperature at 170° C. for the first 24 hours. Presumably, this process causes carbonylation of the cobalt surface and increased FT activity. CO conversion and liquid production were measured at a variety of temperatures between 190° C. and 220° C.

### Example 2

This is the same as Example 1, except that the cobalt metal loading was 10 wt %.

### Example 3

This is the same as Example 1, except that the cobalt metal loading was 15 wt %.

### Example 4

This is the same as Example 1, except that the cobalt metal loading was 20 wt %.

### Example 5

This is the same as Example 1, except that the cobalt metal loading was 26 wt %.

### Example 6

This is the same as Example 1, except that the cobalt metal loading was 35 wt %.

### Influence of Cobalt Loading

The effect of Co loading on catalyst performance was tested with Examples 1-6 with the results shown in Table 2. Tests were conducted at various temperatures and the temperature that gave the largest amount of hydrocarbon product is listed. It is clear that 5% cobalt was not enough to provide a useful amount of liquid hydrocarbons: the best concentration was 20 wt % Co, which gave 1.03 ml/h. The concentration of diesel range hydrocarbons in the hydrocarbon product was 75.3-92.5% at cobalt loadings of 10 wt % cobalt or higher. The highest diesel production rate (0.78 ml/h) was achieved with the trilobe support with 20% cobalt at 70 psia.

TABLE 2
Effect of catalyst loading on performance on Examples 1–6 (trilobes) at 70 psia.

| | Weight % Cobalt | | | | | |
|---|---|---|---|---|---|---|
| | (Example number) | | | | | |
| | 5 (1)* | 10 (2) | 15 (3) | 20 (4) | 26 (5) | 35 (6) |
| Optimum Temperature, ° C. | 220 | 210 | 205 | 200 | 200 | 200 |
| Hydrocarbon Liquid Rate, ml/h | 0.09 | 0.54 | 0.74 | 1.03 | 0.77 | 0.86 |
| Naphtha, wt % | 6.4 | 8.8 | 13.9 | 17.9 | 16.4 | 15.8 |
| Diesel, wt % | 92.5 | 82.8 | 78.3 | 75.3 | 76.8 | 76.8 |
| Light wax, wt % | 1.1 | 8.4 | 7.8 | 6.9 | 6.8 | 7.4 |
| Diesel production, ml/h | 0.08 | 0.45 | 0.58 | 0.78 | 0.59 | 0.66 |
| CO Conversion, mol % | 19.4 | 42.0 | 61.2 | 85.1 | 82.8 | 83.1 |
| C5+ Selectivity, % | 28.6 | 80.6 | 71.3 | 68.0 | 65.1 | 64.3 |
| Cetane number | 81 | 79 | 77 | 76 | 74 | 75 |

*Not part of the present patent application.

### Influence of Pressure

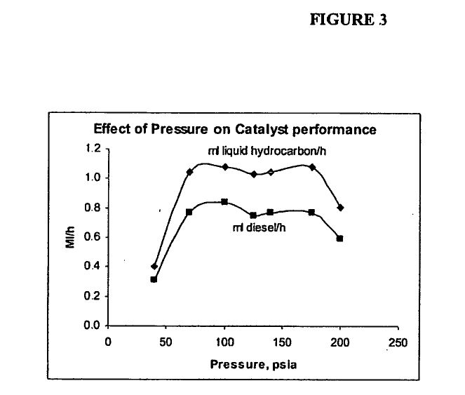

The catalyst in Example 4 was run in the standard testing rig as described above at a temperature of 202.5° C. and at a variety of pressures. Results in Table 3 and FIG. 3 indicate that productivity of the catalyst for production of liquid hydrocarbons is somewhat sensitive to pressure, with the optimum results obtained at pressures between 70 psia and 175 psia. The diesel fraction over that pressure range was fairly constant at 70.8-73.5 weight percent. Surprisingly, the CO conversion decreased as pressure increased in spite of the fact that the gas contact time was greater at higher pressures.

TABLE 3
Effect of pressure on catalyst performance (Example 4 catalyst, 202.5° C.).

| | Pressure, psia | | | | | | |
|---|---|---|---|---|---|---|---|
| | 40 | 70 | 100 | 125 | 140 | 175 | 200 |
| Hydrocarbon Liquid Rate, ml/h | 0.405 | 1.047 | 1.082 | 1.034 | 1.046 | 1.079 | 0.805 |
| Naphtha, wt % | 8.5 | 19.7 | 24.7 | 23.5 | 23.9 | 26.6 | 23.9 |
| Diesel, wt % | 77.8 | 73.5 | 71.9 | 73.1 | 73.4 | 70.8 | 74.1 |
| Light wax, wt % | 13.7 | 6.8 | 3.4 | 3.4 | 2.7 | 2.6 | 2.0 |
| Diesel production, ml/h | 0.32 | 0.77 | 0.78 | 0.76 | 0.77 | 0.76 | 0.60 |
| CO Conversion, mol % | 59.4 | 90.2 | 84.1 | 83.8 | 74.8 | 73.4 | 65.8 |
| C5+ Selectivity, % | 76.6 | 58.1 | 54.4 | 52.5 | 61.3 | 57.7 | 52.0 |

FIG. 3 is a graph showing the effect of pressure on catalyst performance, for a catalyst of Example 4 at 202.5° C.;

### Example 7

This is identical to Example 1, except that the alumina support was CSS-350, obtained from Alcoa, and the cobalt loading was 20 weight percent. This support is spherical with a diameter of 1/16 inch.

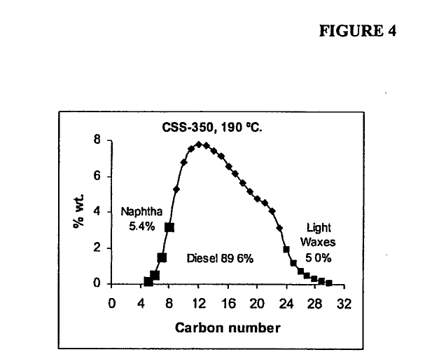

As seen in Table 4, the maximum diesel production rate was achieved at 215° C. and 70 psia. Compared with Catalyst 4, Catalyst 7 gave a lower diesel production rate at its optimum temperature (215° C.), but a higher diesel fraction. FIG. 4 shows the narrow carbon number range in the liquid product at 190° C., with 89.6% in the diesel range. Cetane number was 81. In all graphs of carbon numbers, naphtha is indicated by large squares, diesel by diamonds and light waxes by small squares.

TABLE 4
Performance of Example 7 at various temperatures (CSS-350).

| | Temperature, ° C. | | | | |
|---|---|---|---|---|---|
| | 190 | 200 | 210 | 215 | 220 |
| Hydrocarbon Liquid | 0.55 | 0.58 | 0.64 | 0.70 | 0.68 |
| Rate, ml/h | | | | | |
| Naphtha, wt % | 5.4 | 15.2 | 13.4 | 15.4 | 14.3 |
| Diesel, wt % | 89.6 | 76.8 | 82.0 | 77.4 | 81.3 |
| Light wax, wt % | 5.0 | 8.0 | 4.6 | 7.2 | 4.4 |
| Diesel production, ml/h | 20.1 | 47.2 | 45.2 | 53.8 | 49.5 |
| Average Molecular | 194.9 | 170.2 | 171.2 | 164.8 | 168.3 |
| Weight | | | | | |
| CO Conversion, mol % | 47.8 | 53.4 | 81.6 | 93.8 | 100.0 |

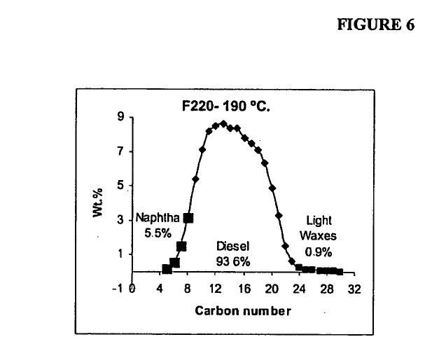

FIG. 6 is a graph showing the effect of Carbon number distribution on the % weight production of liquid fuels and/or waxes, at 190° C. at 70 psia using an F-220 alumina support (ie Example 9);

### Example 8

This is identical to Example 1, except that the alumina support was LD-5, obtained from Alcoa, and the cobalt loading was 20 weight percent. This support is spherical with a mesh size distribution of 7/14.

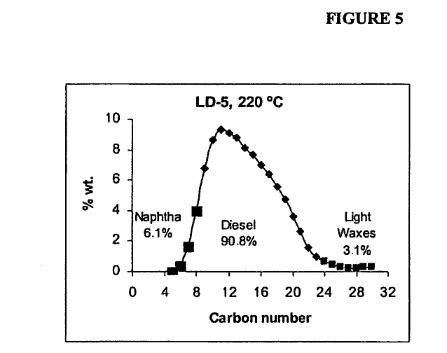

Example 8 was tested at 70 psia and two temperatures. Table 5 shows that the optimum temperature was 220° C. and the hydrocarbon liquid was 6.1% naphtha, 90.8% diesel and 3.1% light waxes. FIG. 5 shows the carbon number distribution. Cetane number was 83. All cetane numbers were calculated from the total liquid hydrocarbon produced and so the numbers quoted are lower than for the diesel fraction alone, which was 91 in this case. Likewise, Average Molecular Weight is for the total liquid.

TABLE 5
Performance of Catalyst 8 (LD-5) versus temperature at 70 psia.

| | | |
|---|---|---|
| Temperature, ° C. | 210 | 220 |
| Hydrocarbon Liquid Rate, ml/h | 0.6 | 0.65 |
| Naphtha, wt % | 8.6 | 6.1 |
| Diesel, wt % | 78.4 | 90.8 |
| Light wax, wt % | 13.0 | 3.1 |
| Diesel production, ml/h | 0.43 | 0.55 |
| Average Molecular Weight | 186.5 | 184.0 |
| CO Conversion, mol % | 53.3 | 69.3 |

FIG. 5 is a graph showing carbon number distribution for Example 8 (LD-5) at 220° C., 70 psia.

### Example 9

This is identical to Example 1, except that the alumina support was F-220, obtained from Alcoa, and the cobalt loading was 20 weight percent. F-220 is a spherical support with a mesh size distribution of 7/14.

Example 9 catalyst was tested at 70 psia. As shown in Table 6 and FIG. 6, the 190° C. hydrocarbon product contained 99.1% naphtha plus diesel. Diesel itself was at 93.6%. There was very little light wax. Cetane number was 81.

TABLE 6
Performance of Example 9 (F-220) at various
temperatures (pressure 70 psia).

| | | | | |
|---|---|---|---|---|
| Temperature, ° C. | 190 | 200 | 210 | 215 |
| Hydrocarbon Liquid Rate, ml/h | 0.465 | 0.757 | 0.8 | 0.733 |
| Naphtha, wt % | 5.5 | 9.2 | 20.1 | 21.5 |
| Diesel, wt % | 93.6 | 88.5 | 77.0 | 74.7 |
| Light wax, wt % | 0.9 | 2.3 | 2.9 | 3.8 |
| Diesel production, ml/h | 0.41 | 0.62 | 0.53 | 0.47 |
| Average Molecular Weight | 188.2 | 181.4 | 157.7 | 154.1 |
| CO Conversion, mol % | 50.0 | 72.2 | 94.7 | 92.2 |
| Cetane number | 81.0 | 76.0 | 67.0 | 65.0 |

FIG. 6 is a graph showing carbon number distribution for Example 9 (F-220) at 190° C., 70 psia.

### Example 3

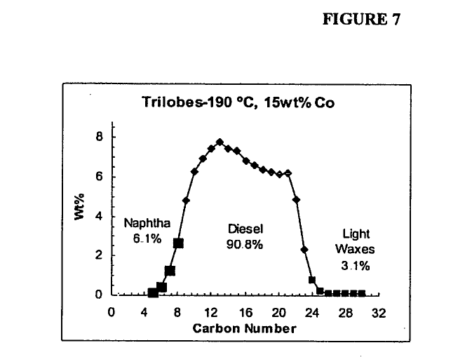

FIG. 7 shows the carbon number distribution for catalyst Example 3 (trilobe) at 190° C. A very narrow distribution was obtained having no heavy wax. Diesel was 90.8%, naphtha 6.1% and light waxes 3.1%. Cetane number was very high at 88.

### Example 10

This is identical with Example 4, except that the promoter was ruthenium rather than rhenium.

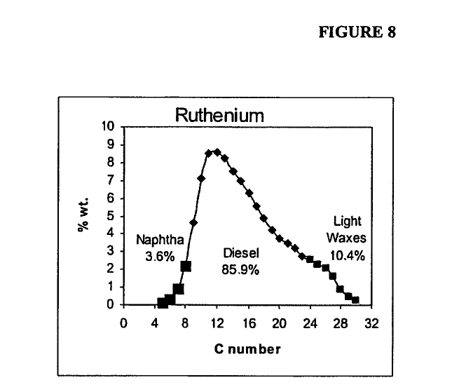

Data in Table 7 and FIG. 8 show that the use of ruthenium catalyst promoter instead of rhenium also provides a narrow distribution of hydrocarbons with 85.9% in the diesel range having an overall cetane number of 80.

TABLE 7
Performance of ruthenium promoter, LD-5 alumina support.

| | |
|---|---|
| Temperature, | 210.3/72 |
| ° C./Pressure, psia | |
| Conv CO to CH4, % | 6.43 |
| Conv CO to C2H6, % | 1.47 |
| Conv CO to C3H8, % | 1.74 |
| Conv CO to C4H10, % | 2.55 |
| Conv CO to CO2, % | 2.91 |
| CO unreacted, % | 38.28 |
| Total Conv CO, % | 61.72 |
| CO converted to C5+, % | 46.62 |
| Hydrocarbon Liquid Rate, ml/h | 0.731 |
| Diesel production, ml/h | 0.451 |
| Naphtha, % | 3.6 |
| Diesel, % | 85.9 |
| Light waxes, % | 10.4 |
| Cetane number | 80 |
| Average MW | 199.6 |

FIG. 8 is a graph showing the effect of carbon number distribution on the % weight production of liquid fuels and/or waxes, for the catalyst of Example 10 using ruthenium promoter.

### Example 11

This was identical with Example 3, except that Aerolyst 3038 silica catalyst support from Degussa was used instead of alumina.

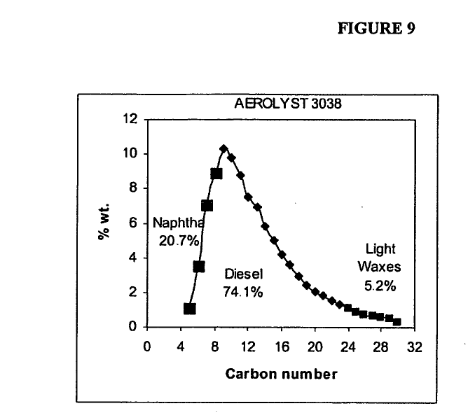

FIG. 9 is a graph showing the effect of carbon number distribution on the % weight production of liquid fuels and/or waxes, for ruthenium promoter and LD-5 alumina support, using an Aerolyst 3038 silica catalyst support instead of aluminas a graph showing the effect of carbon number distribution on the % weight production of liquid fuels and/or waxes, for ruthenium promoter and LD-5 alumina support, using an Aerolyst 3038 silica catalyst support instead of alumina.

The hydrocarbon liquid production rate was 0.55 ml/h at 210° C. The carbon distribution curve shown in FIG. 9 demonstrates a narrow distribution with a high diesel cut.

### SUMMARY

The Examples 1-11 in this disclosure show that a narrow distribution of hydrocarbons, mainly in the diesel range, is obtained. FIG. 9 compares this result with expectations from the Anderson-Shultz-Flory (A-S-F) carbon number distribution based on chain growth. The liquid hydrocarbon product of the Examples is more valuable than the broad A-S-F type of product because it can be used directly as a diesel-blending stock to increase cetane number and decrease sulphur content of petroleum diesels. Because the present process is a simple once-through process, it entails low capital cost.

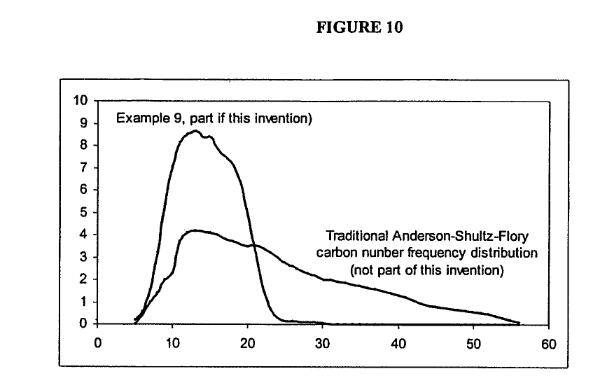

FIG. 10 is a graph showing a comparison of Example 9 carbon distribution with a traditional Anderson-Shultz-Flory distribution.

1. A Fischer-Tropsch process operating at less than 200 psia, using an air autothermal reformer, and having a CO conversion of at least 65% and providing a diesel yield greater than 60% by weight in a single-pass fixed-bed Fischer-Tropsch reactor using a catalyst, said catalyst having a metallic cobalt loading greater than 5% by weight and rhenium loading of less than 2% by weight on a catalyst support material selected from the group of catalyst support materials comprising alumina, zirconia, and silica.

2. The process of claim 1 wherein the Fischer-Tropsch catalyst support material is comprised of alumina.

3. The process if claim 1 having a lead gas, wherein selective membranes or molecular sieves are employed to remove hydrogen from the feed gas.

4. The process of claim 1 wherein the cobalt catalyst loading is greater than 6 weight %.

5. The process of claim 1 wherein the operating pressure is less than 100 psia.

6. A Fischer-Tropsch process as claimed in claim 1, said reactor further having a promoter, wherein said promoter comprises a promoter selected from the group of promoters consisting of ruthenium and rhenium or mixtures thereof.

7. A Fischer-Tropsch process operating at less than 200 psia, using an oxygen autothermal reformer, and having a CO conversion of at least 65% and providing a diesel yield greater than 60% by weight in a single-pass fixed-bed Fischer-Tropsch reactor using a catalyst with a metallic cobalt loading greater than 5% by weight and a rhenium loading of less than 2% by weight on a catalyst support selected from the group of catalyst supports comprising alumina, zirconia and silica.

8. The process of claim 7 wherein the Fischer-Tropsch catalyst support is comprised of alumina.

9. The process of claim 7 having a tailgas from the reformer, wherein the tailgas is partially recycled to the reformer.

10. The process of claim 7 further having a feed gas wherein selective membranes or molecular sieves are employed to remove hydrogen from the gas.

11. The process of claim 7 wherein the cobalt catalyst loading is greater than 6 weight %.

12. The process of claim 7 wherein the operating pressure is less than 100 psia.

13. A Fischer-Tropsch process of claim 7 said reactor further having a promoter, wherein said promoter comprises a promoter selected from the group of promoters consisting of ruthenium and rhenium or mixtures thereof

14. A Fischer-Tropsch process operating at less than 200 psia, using an oxygen steam reformer, and having a CO conversion of at least 65% and providing a diesel yield greater than 60% in by weight in a single-pass fixed-bed Fischer-Tropsch reactor using a catalyst with a metallic cobalt loading greater than 5% by weight and rhenium loading of less than 2% by weight on a catalyst support selected from the group of catalyst support comprising alumina, zirconia, and silica.

15. The process of claim 14 wherein the Fischer-Tropsch catalyst support is comprised of alumina.

16. The process of claim 14 further having a feed gas, wherein selective membranes or molecular sieves are employed to remove hydrogen from the feed gas.

17. The process of claim 14 having a tailgas from the reformer, wherein some or all of the tailgas is burned to provide heat to the reformer.

18. The process of claim 14 wherein the cobalt catalyst loading is greater than 6 weight %.

19. The process of claim 14 wherein the operating pressure is less than 100 psia.

20. A Fischer-Tropsch process of claim 14, said reactor further having a promoter, wherein said promoter comprises a promoter selected from the group of promoters consisting of ruthenium and rhenium or mixtures thereof

21. A Fischer-Tropsch process operating at less than 200 psia, using an air or oxygen partial oxidation reformer, and having a CO conversion of greater than 65% and providing a diesel yield greater than 60% by weight in a single-pass fixed-bed Fischer-Tropsch reactor using a catalyst with a metallic cobalt loading greater than 5% by weight and rhenium loading of less than 2% by weight on a catalyst support selected from the group of catalyst supports comprising alumina, zirconia, and silica.

22. The process of claim 21 wherein the Fischer-Tropsch catalyst support is comprised of alumina.

23. The process of claim 21 having a feed gas, wherein selective membranes or molecular sieves are employed to remove hydrogen from the feed gas.

24. The process of claim 21 wherein the cobalt catalyst loading is greater than 6 weight %.

25. The process of claim 21 wherein the operating pressure is less than 100 psia.

26. A Fischer-Tropsch process of claim 21, said reactor further having a promoter, wherein said promoter comprises a promoter selected from the group of promoters consisting of ruthenium and rhenium or mixtures thereof.
